(19) United States
(12) Patent Application Publication
(10) Pub. No.: US 2003/0108617 A1
(43) Pub. Date: Jun. 12, 2003

(54) Use of a formulation made of or containing at least one dissimilated milk serum

(76) Inventors: Ulrich Leithe, Berlin (DE); Adrianus Mattheus Maria Zijerveld, Vleuten (NL)

Correspondence Address: Stephan A Pendorf, Pendorf & Cutliff, PO Box 20445, Tampa, FL, 33622-0445, US

(21) Appl. No.: 10/312,350

(22) PCT Filed: Jun. 25, 2001
(86) PCT No.: PCT/EP01/07202

(30) Foreign Application Priority Data
Jun. 28, 2000 (DE) 100 31 506.2
Jun. 28, 2000 (DE) 100 31 507.0
Jun. 28, 2000 (DE) 100 31 505.4
Jun. 28, 2000 (DE) 100 31 504.6
Jul. 3, 2000 (DE) 100 32 243.3
Jul. 11, 2000 (DE) 100 33 560.8
Jul. 11, 2000 (DE) 100 33 559.4
Jul. 11, 2000 (DE) 100 33 558.6
Feb. 20, 2001 (DE) 101 07 859.5

(51) Int. Cl. A61K 35/20

(52) U.S. Cl. 424/535

(57) ABSTRACT

Use of a composition consisting of or with a content of at least a dissimilated milk serum to improve the tolerance of the human or animal organism.

### BACKGROUND OF THE INVENTION

1. Field of the Invention

The invention relates to a novel use of a composition consisting of or with a content of at least a dissimilated milk serum.

2. Description of the Related Art

EP 0 357 162 B1 discloses a method of modifying whey in which the whey is at least partially eliminated by fermentation using suitable lactic acid bacteria lactose. Once whey has been reacted in this way, it can be used in cleansing products, body cleansing agents, personal hygiene products, such as creams, lotions, ointments, etc., or pharmaceutical preparations. As examples of the use of the modified whey in the pharmaceutical sector, mention may be made of preparations to improve, sustain and/or restore the immune system of the human skin and mucous membrane. No specific medical indications are mentioned, any more than the effect on the tolerance of the human or animal organism.

Securing and adapting the optimum dissimilation processes and tolerances of the variable standards for regulating continuous biological processes in the metabolism is an important precondition for keeping the human or animal organism healthy. One precondition for this in turn could be achieved by raising the organism's tolerance to (internal and/or external) damage or influences which disturb these processes. In this way, it would be possible to achieve both a better adaptability on the part of the organism to changed (internal and/or external) situations or a potential for improved prevention or cure. So far, however, no simple possibility is known for achieving a targeted improvement or, even more preferably, optimisation of the tolerance of the human or animal organism.

Numerous diseases are known in which the course of the biological processes is disturbed, which then contributes to the development of disease symptoms.

A particularly clear example can be found in a number of skin disorders.

So far, a wide variety of approaches have been adapted for the treatment of psoriasis, ichthyosis, atopic and orthoergic eczema, acne and etiologically similar skin disorders.

With the above-mentioned skin disorders, apart from light therapy, i.e. irradiation with ultraviolet light, it is common to use above all creams and ointments containing corticosteroids. Corticosteroids are hormones and have a wide variety of negative side-effects, especially when used over a long period, e.g. the skin may become thinner, and there may be circulatory disorders. Furthermore, the use of corticosteroids requires a constant increase in the dosage and/or strength of the medication in order to keep the disease under control. Diseases such as psoriasis are considered incurable with existing drugs.

Another case in which a disturbance in the biological processes can lead to diseases is cardiovascular diseases.

Plaque deposits in the coronary vessels are among the most frequent and serious causes of cardiovascular diseases. The standard therapy today consists in the invasive expansion of the coronary vessels by dilatation and the insertion of stents, accompanied by the need to adhere to a particular diet and take medication. In cases where plaque deposits are already highly advanced, it can also be necessary to place bypasses. Cardiovascular diseases, which are among the most common cause of death nowadays, and especially plaque deposits, can therefore only be treated by invasive surgery and/or medication.

Disturbances in the biological processes can also be at least one of the causes of diseases of the gastro-intestinal tract and of rheumatic and rheumatoid disorders.

One special group of gastro-intestinal disorders is inflammatory diseases of the intestines. Particular mention can be made here of Crohn's disease and the various forms of colitis. Crohn's disease, also known as enteritis regionalis, is a chronic form of inflammation of the lower segments of the small intestine which is found above all in younger adults and which usually occurs in episodes that frequently spread to the large intestine. The various kinds of colitis involve acute or chronic inflammation mucous membrane of the large intestine, often extending throughout and involving even deeper layers of the intestine wall. In order to treat these diseases, it is customary to use immunosuppressants, such as prednisone and cyclosporines.

Rheumatic and rheumatoid diseases constitute a major health problem throughout the world. A wide variety of rheumatic and rheumatoid diseases are known, such as inflammatory rheumatism of the joints and soft tissue, muscular rheumatism, rheumatism of the joints (idiopathic, infectious, allergic, degenerative), rheumatoid arthritis and psoriasis arthritis. These diseases, which are frequently accompanied by severe pain and restrictions on movement, are usually treated with powerful medication, such as methotrexate or immunosuppressants (often corticosteroids), which often involve major side-effects and other impairments.

Another group of diseases in whose development the disturbance of biological processes in the metabolism appears to play a role is a number of mental disturbances.

Manic-depressive diseases are endogenous psychoses in which depressive and manic phases alternate. Because of the peculiarities of this syndrome, treatment with extremely potent neuroleptic drugs is necessary in the acute phase, while antidepressants are required in the depressive phase. As a consequence of this, manic-depressive diseases have to be constantly monitored by a physician, so that the type and dosage of the medication can be adapted to the phase concerned. Furthermore, many neuroleptic drugs and antidepressants exhibit undesirable side-effects.

Schizophrenia is a psychosis involving the loss of the structural correlation of the personality and a split in thought, affect and experience. The etiogenesis of this disorder is unknown. The disorder develops in very varied ways (it may be acute or chronic or appear in episodes). The conventional treatment of this disorder is with psychotropic drugs in combination with psychotherapy and psychoanalysis, and in more recent times occasionally also with anti-inflammatory agents such as prednisone. The use of drugs of this kind often results in severe side-effects and other impairments, however.

Since, in the case of the above-mentioned disorders, the use of various drugs often involves severe side-effects, there is therefore a need for the provision of an alternative to the existing methods of treating them, avoiding these disadvantages.

### SUMMARY OF THE INVENTION

In accordance with the invention, this problem is soled by the use of a composition consisting of or with a content of at least a dissimilated milk serum to improve the tolerance of the human or animal organism.

In preferred embodiments of the invention, the composition is used for the treatment, prevention and prophylaxis von psoriasis, ichthyosis, atopic and orthoergic eczema, acne and etiologically similar dermatological disorders, and also skin infections, skin injuries or skin irritations, cardiovascular diseases, inflammatory intestinal diseases, especially Crohn's disease and/or colitis, rheumatic and rheumatoid diseases, manic-depressive disorders and schizophrenia.

In a preferred embodiment of the invention, the composition is used as an additive in foodstuffs or animal feed.

In a further embodiment of the invention, it is provided for the composition to be used in order to manufacture a pharmaceutical.

It is also provided, in accordance with the invention and in the case of a medical indication, for the composition to be used in combination, simultaneously or consecutively, with the active agents that can be employed for the indication concerned.

As the starting product in manufacturing the dissimilated milk serum, a milk serum can preferably be used which has been obtained from cow's milk, sheep's milk, goat's milk, horse milk and/or camel milk.

It is preferable to use whey which has been obtained as a by-product in the production of cheese, cottage cheese or yoghurt.

The dissimilated milk serum is preferably obtained by fermentation with one or more micro-organisms selected from the group consisting of lactobacilli, leuconostoc, streptococci and other lactic acid bacteria.

In connection with the present application, milk serum is understood to mean a liquid obtained from milk after the at least partial separation of fat and casein. One form of milk serum is the whey which is obtained as a by-product in the production of cheese, cottage cheese or yoghurt.

Dissimilation for the purposes of the present application is understood to mean general methods in which substances with higher molecular weights are converted into lower molecular-weight substances, generating energy in the process. One example of this is fermentation by micro-organisms.

Totally surprisingly, it was found that dissimilated milk sera—alone or as a component of a suitable composition—are outstandingly appropriate for adjusting the tolerance of the human or animal organism, including micro-organisms, to its optimum. It has become apparent that this can be exploited in a variety of ways for the prevention, prophylaxis or therapy of diseases, but also generally in order to strengthen and fortify the organism. Specifically, it has become apparent that dissimilated milk sera can be successfully used to treat the above-mentioned dermatological disorders, skin infections, skin injuries or skin irritations. In the same way, dissimilated milk sera can be successfully used to prevent the progression of plaque deposits in blood vessels and are thus suitable for the treatment, prevention and prophylaxis of cardiovascular diseases. They are also effective in the treatment of inflammatory intestinal diseases and thus also in the prevention and prophylaxis of such diseases. Furthermore, it has been found that dissimilated milk sera can replace the drugs used hitherto to treat rheumatic and rheumatoid disorders or, when used in combination, can at least reduce the dosage of those drugs. In the treatment, prevention or prophylaxis of manic-depressive disorders or schizophrenia too, it has become apparent that, by using dissimilated milk sera, it is at least possible to reduce the existing medication substantially. It goes without saying, however, that the invention is not restricted to the diseases mentioned.

Without wishing to be committed to this theory, it is currently considered probable that the treatment successes with dissimilated milk serum are connected with the improvement or optimisation of the organism's tolerance, which has now been proven. A controlled dissimilation of milk sera clearly yields balanced metabolite complex substrates, with which the above-mentioned effects can be achieved.

The composition used in accordance with the invention is preferably prepared from milk serum obtained from the milk of cows, sheep, goats, horses, camels or other mammals. Whey is particularly suitable, which is obtained in the production of cheese or cottage cheese, which takes the form of acidification and/or the addition of rennin.

It should be pointed out that the method of obtaining milk serum is not restricted to the traditional process of cheese production, but that any process can be used by which the fat and casein are at least partially, preferably substantially, and most preferably completely separated from milk.

The currently preferred method for the dissimilation of milk serum is microbial conversion, especially fermentation with one or more micro-organisms under anaerobic or micro-aerophilic conditions. For the microbial dissimilation of milk serum, it is particularly suitable to use lactobacilli, such as Lactobacillus acidophilus, Lactobacillus bulgaricus, Lactobacillus lactis, Lactobacillus helveticus and Lactobacillus caseii, leuconostoc, streptococci, such as Streptococcus lactis and Streptococcus thermophilus, and other lactic acid bacteria, but also yeasts and other fungi.

The fermentation temperature and the duration of the fermentation process can be varied within a certain range, depending on the desired characteristics of the dissimilated milk sera and the starting materials and micro-organisms used. The preferred fermentation temperature is usually in the range from 25 to 40° C. and the duration of fermentation is preferably 1 to 30 days.

It goes without saying that other methods for the dissimilation of milk serum are conceivable and can be used for the purposes of the present application, provided that it is ensured that the intended purpose, namely the conversions of higher molecular-weight substances into lower molecular-weight substances, generating energy in the process, is achieved. Methods of this kind can, for example, also take place in a purely chemical manner.

The dissimilated milk serum is preferably formulated for use in accordance with the invention in such a way that it used as an additive in foodstuffs or animal feed. In the case of medical indications, however, it can also be used as a preparation for oral or other (e.g. intravenous or topical) administration. In the process, the dissimilated milk serum or the preparation containing it can in some cases be administered alone, or in other cases preferably in combination with other drugs for the medical indications concerned. Especially when used in combination with other drugs, it has become apparent that the dosage of the latter can at least be reduced considerably, which is particularly interesting especially when drugs of this kind produce significant side-effects.

The dissimilated milk serum can also be employed to improve the activity of microorganisms, e.g. for use in sewage treatment plants.

The non-concentrated fermented milk serum can be used directly and without any further processing, i.e. in liquid form. Even though the micro-organisms used may still be present in the finished formulation, the dissimilated milk serum can nevertheless also be used in pasteurised or sterilised form. Furthermore, it is possible to convert the dissimilated milk serum into a viscous or even solid product by means of concentration. For concentration purposes, it is, for example, possible to use membrane filtration processes or the gentle removal of water vapour under reduced pressure at mild temperatures, such as below 40° C.

The concentrated product can be even further dried in a suitable manner, e.g. freeze-dried, and optionally pulverised. From appropriate material and using suitable procedures, it is also possible to produce tablets for oral administration, which can also be given a coating resistant to gastric juices if so desired.

The dosage of dissimilated milk serum to be administered, or of the preparation containing it, can vary over a wide range, depending on the field of use, symptoms, age and condition of the patient, stage of the disease, supplementary medication, methods of application, form of application, etc. For the person skilled in the art concerned, however, it is possible with no difficulty to find a suitable dosage; for the indications described in more detail in the examples, the quantities specified there may serve as an indication.

### EXAMPLE 1

### Dissimilation of Milk Serum

In order to produce a possible dissimilated milk serum, the whey from cow milk obtained in the course of cheese production is poured straight into fermentation tanks. The temperature of the whey is about 30° C., the pH 5.8 and the lactose content 47%. This whey is then inoculated with an advance culture of Lactobacillus acidophilus. The tanks are sealed with fermentation caps and incubated for 25 days at 30° C.

The milk serum dissimilated in this way has a pH of 3.4 and a lactose content of 0.35% in a highly lactic acidic environment. The population of micro-organisms is clearly dominated by Lactobacillus acidophilus.

The dissimilated milk serum produced in this way can be used, without any further treatment, for the uses mentioned. Concentration in order to reduce the volume by a factor of 5, preferably 10, is possible by removing the water in a vacuum at a maximum temperature of 40° C.

### EXAMPLE 2

### Effect of the Composition of the Invention on the Tolerance of the Organism

In order to test the efficacy of the composition of the invention for the use in accordance with the invention, a dissimilated milk serum as produced in Example 1 was used. The influence of the composition of the invention was tested in vivo on the Stratum corneum of the human skin. The measurements were performed with infrared spectrometry. For this purpose, a medium-spectrum infrared spectrometer (Biorad Excalibur) with a flat crystal was used. The angle of incidence was 45°.

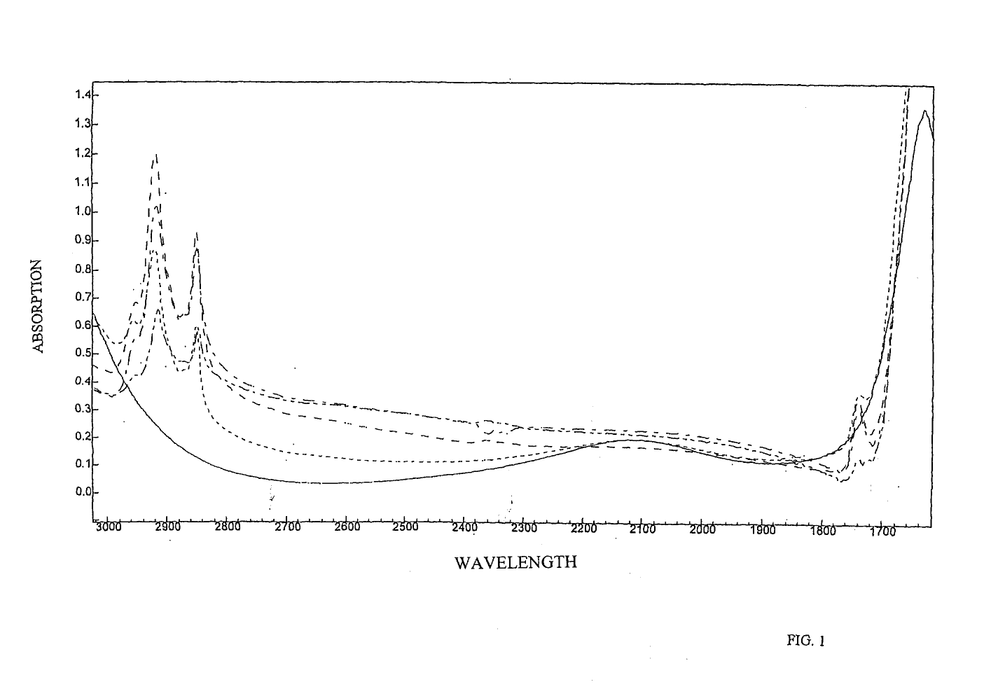

Measurements were performed on a total of 20 subjects (on the left forearm in each case). The results can be seen from FIGS. 1 to 4.

The measurements were first performed on untreated, undamaged skin. FIG. 1 shows, by way of example, the infrared spectra of skin from some subjects in the wavelength range from 3,000 to 1,600. The infrared spectra in all the subjects tested exhibit a similar development in this range, i.e. pronounced peaks in the range from 3,000 to 2,800 and above approx. 1,700. In the range between 2,700 and 1,800, the curve is basically flat, with no significant peaks.

In a next step, the minimum erythema dose (MED) was determined in the subjects, i.e. the lowest does of UV B radiation that causes a skin erythema after approx. 8 h.

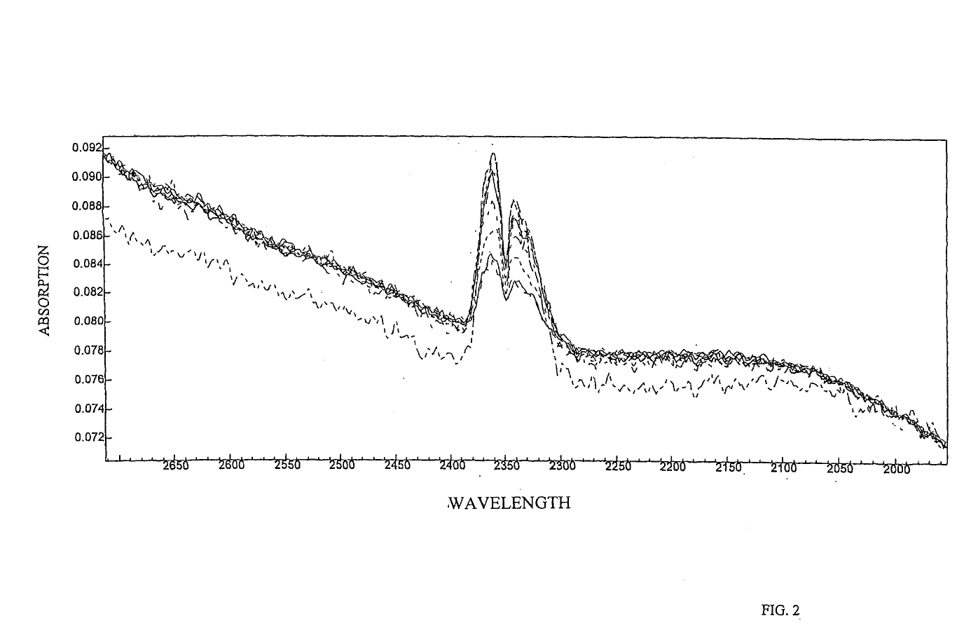

After UV irradiation of the skin areas with 70% MED and further measurement with the infrared spectrometer, a uniformly significant curve appeared in the wavelength range between 2,450 and 2,250, as is shown, by way of example, in FIG. 2. The corresponding peaks can be attributed to the formation of cis-urocanic acid from the trans-isomer present in the skin cells, as a result of the UV irradiation. It is known that trans-urocanic acid is a UV absorber formed naturally in the skin, which is rearranged into the corresponding cis-isomer in the event of UV irradiation. It should be pointed out that the curve in the wavelength range of FIG. 2 is virtually identical both immediately after the irradiation and even after two weeks.

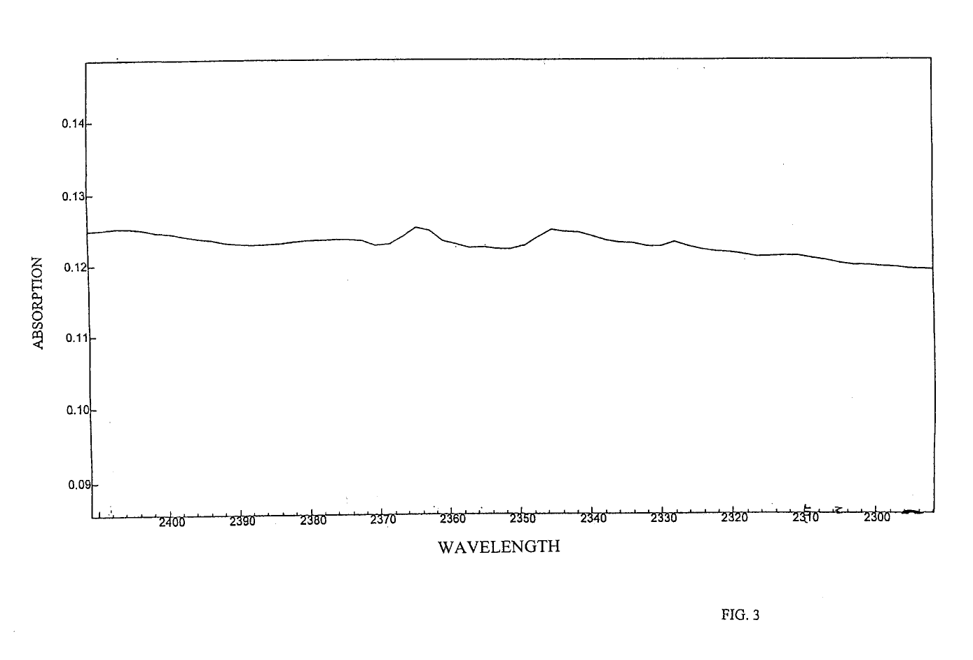

In ten subjects, the irradiated skin regions were treated twice a day over a period of two weeks. A further measurement of the treated skin regions produced the spectrum of FIG. 3, in which it can be seen that the peaks have virtually disappeared, i.e. a return to a substantially flat curve can be observed, which permits the conclusion to be drawn that the situation in the skin has returned to normal as a result of the treatment with the composition of the invention.

The MED value was again determined in all the subjects. Whereas in the subjects who had not been treated with the composition of the invention the same MED values were found as before the first UV irradiation, in the group of subjects who had been treated with the composition of the invention distinctly higher MED values were found, i.e. the skin's tolerance to UV irradiation had been increased significantly as a result of the treatment with the composition of the invention.

The areas of skin concerned were now irradiated again with 70% MED, i.e. in the case of the subjects treated with the composition of the invention it was a higher dose in absolute terms. After the further UV irradiation, the infrared spectra apparent from FIG. 4 were found, the lower, unbroken line representing a curve in subjects who had been treated with the composition of the invention, whereas the upper, broken line shows the result in subjects who had not been treated with the composition of the invention.

Whereas, in the untreated subjects, substantially the same curve can be seen as after the first UV irradiation, it can be seen that the skin of subjects treated with the composition of the invention does not exhibit the change which is typical of UV irradiation. On the contrary, the curve is substantially the same as before the UV irradiation. Without wishing to be committed to any theory, it is currently supposed that skin treated with the composition of the invention is capable of tolerating UV light to a greater extent, without the formation and/or conversion of trans-urocanic acid playing a role in the process. It should be pointed out that measurements of the composition of the invention have proven that it does not contain any components which can be directly regarded as UV absorbers. The effects observed can therefore indeed be attributed to an increase in the skin's tolerance to UV irradiation. This, seen together with the successes described in more detail below in the treatment of various diseases permits the conclusion to be drawn that the use of the composition of the invention leads to an improvement, or optimisation, of the organism's tolerance.

### EXAMPLE 3

### Use of the Composition of the Invention to Treat Skin Disorders

In all the examples, a preparation was used which can be produced in accordance with Example 1.

### EXAMPLE 3a

Nine persons with an atopic eczema who had suffered an allergy to cow milk in younger years, and who had a positive IgE RAST to cow milk, were tested for a possible allergic skin reaction by contact with the preparation (without preservatives and scent). The preparation was contacted with the skin on the inside of the subjects' forearms for 48 hours under occlusion, by means of a patch test. In none of the subjects was a type IV allergic reaction found at the end of the test. After that, a scratch test was performed on the inside of the forearm. In none of the subjects was a type I allergic reaction found.

### EXAMPLE 3b

On eight subjects with chronic and stable psoriasis on the extensor side of the elbow, which had to be left untreated for more than three weeks before the beginning of the study, the preparation was applied over a period of three weeks on the extensor side of their left elbow. The right elbow was left completely untreated. After one week, none of the subjects notices any beneficial effect. Towards the end of the third week, a visible and perceptible improvement in the skin abnormalities was observed in six of the eight subjects, compared to the beginning of the study and compared to their untreated elbow.

### EXAMPLE 3c

For this study, nine individuals with chronic orthoergic eczema of the hands (“dishwashing hands”) were selected, whose orthoergic eczema had not been treated for longer than three weeks, and who had been instructed to continue their daily chores unchanged. Within a matter of days after the beginning of treatment by the topical application of the preparation, all nine patients reported a pronounced subjective and perceptible improvement. All nine patients completed the three-week study, and it was found that the positive effect had remained unchanged over those three weeks. A dermatological examination after those three weeks found a distinct reduction in the desquamation on the fingers and a distinct impression of more supple skin.

### EXAMPLE 3d

Nine individuals with stable chronic atopic eczema in the folds of the elbow were instructed to apply the preparation twice a day for three weeks to the left fold of the elbow and to leave the right fold of the elbow untreated. After one week, all the individuals found that the preparation initially caused a stinging feeling in the fold of the elbow. After the third week, a gradual improvement effect was found in seven individuals. What was very striking here was the reduction in the itching. These seven individuals wanted to continue the treatment with the preparation even after the third week.

### EXAMPLE 3e

Six individuals with atopic eczema on both forearms were treated for three weeks on the first three days of the week on both forearms with a grade III corticosteroid and on the following four days of the week with an emulsion (liquid paraffin and soft white paraffin in equal quantities). The right forearm was always to be rubbed with the preparation half an hour beforehand. If possible, the four days without treatment with corticosteroids were to be extended by one further day every two weeks. It turned out that only the forearm treated with the preparation could tolerate a longer pause between the corticoid applications: in all the subjects, this period increased from four to five and then to six days.

### EXAMPLE 4

### Use of the Composition of the Invention in the Treatment of Dermatological Infections, Skin Irritations and Skin Injuries

The preparation, which is produced in accordance with Example 1, can also be used successfully on a wide variety of dermatological infections, skin irritations and skin injuries.

It has been found, for example, that in the case of severe skin irritations because of excessive UV radiation (sunburn) external treatment with the preparation of the invention leads to a more rapid alleviation of the symptoms, and especially that it causes the reddening of the skin and the feeling of tautness to subside more rapidly.

In a wide variety of dermatological infections or skin injuries as well, more rapid healing was observed. This applies not only to normal cuts and grazes, but in particular also to more serious skin injuries such as ulcus crusis (leg ulcers) and burns. A distinct acceleration of the healing process was observed.

### EXAMPLE 5

### Use of the Composition of the Invention for the Treatment of Arteriosclerosis

The study was conducted on 8 patients suffering from advanced arteriosclerosis with significant plaque deposits. In all the patients, a balloon dilatation had been performed shortly before the beginning of the study and a stent had been inserted.

At the same time as their usual medication, the patients were given a daily dose of 200 ml undiluted dissimilated milk serum, produced in accordance with Example 1, which they took spread out over the day.

In all the patients, a distinct stabilisation of their condition was observed. After dilatation and the insertion of stents, plaque usually tends to form again after a certain time, which requires a further operation. It was possible to extend this period considerably. Follow-up examinations after 6 and 12 months respectively did not find any signs of significant new plaque deposits in the coronary vessels.

### EXAMPLE 6

### Use of the Composition of the Invention for the Treatment of Crohn's Disease

The study was conducted on 10 patients, who were adults aged between 20 and 40. The patients were suffering from Crohn's disease and were being treated with the immunosuppressant prednisone.

From the beginning of the study, all the patients were given a daily dose of 200 ml undiluted dissimilated milk serum, produced in accordance with Example 1, at the same time as their usual prednisone medication, and took it spread out over the day. After 6 weeks, the prednisone dose was gradually reduced. After 12 weeks, it was possible to discontinue the immunosuppressant completely.

Both during the period of the gradual reduction in the prednisone dose and after the complete discontinuation of the immunosuppressants, no reoccurrence of the symptoms of the disease was observed.

Similar results were found with the same scheme of treatment, i.e. gradual reduction in the dose of immunosuppressant until its complete discontinuation, in patients suffering from colitis ulcerosa.

### EXAMPLE 7

### Use of the Composition of the Invention fro the Treatment of Articular Rheumatism and Arthritis

The study was conducted on 10 patients, who were adults aged from 40 to 70. 5 patients were suffering from articular rheumatism, 5 more from rheumatoid arthritis. All the patients were being given methotrexate and/or immunosuppressants.

From the beginning of the study, all the patients were given a daily dose of 200 ml undiluted dissimilated milk serum, produced in accordance with Example 1, at the same time as their usual medication, and took it spread out over the day. After 6 weeks, the dose of medication was gradually reduced. After 12 weeks, the usual medication was discontinued completely.

Both during the period of the gradual reduction in the dose of medication and after its complete discontinuation, a stabilisation, and in three cases a distinct improvement in the patients' condition was observed.

### EXAMPLE 8

### Use of the Composition of the Invention for the Treatment of Manic-Depressive Disorders

The study was conducted on 8 patients. The patients were suffering from manic-depressive disorders and were being treated with Haldol in the acute phase and Clomipramine in the depressive phase.

From the beginning of the study, all the patients were given a daily dose of 200 ml undiluted dissimilated milk serum, produced in accordance with Example 1, at the same time as their usual medication, and took it spread out over the day. After 10 days, the dose of their usual medication was gradually reduced and discontinued completely after 3 weeks.

Both during the period of the gradual reduction in the dose of the usual medication and after its complete discontinuation, no reoccurrence of the symptoms of the disorder was observed. After 3 to 4 weeks without the usual medication, reduced symptoms recurred in one patient in the manic phase, so that that patient was changed to additional drug treatment (reduced to ⅓ of the usual dose) with Haldol.

### EXAMPLE 9

### Use of the Composition of the Invention for the Treatment of Schizophrenia

The study was conducted on 7 patients. The patients had been suffering for some length of time from symptoms of the schizophrenia syndrome and were being treated with appropriate psychotropic drugs (especially Haloperidol) or anti-inflammatory agents such as prednisone.

From the beginning of the study, all the patients were given a daily dose of 200 ml undiluted dissimilated milk serum, produced in accordance with Example 1, at the same time as their usual medication, and took it spread out over the day. After 2-3 weeks, the dose of their usual medication was gradually reduced and discontinued completely after about 2 months.

Both during the period of the gradual reduction in the dose of the usual medication and after its complete discontinuation, no reoccurrence of the symptoms of the disorder was observed. About 3 weeks after discontinuation of the psychotropic drug, reduced symptoms recurred in one patient. With that patient, the usual drug treatment (though reduced to ¼ of the previous dose) was resumed in addition.

The features of the invention disclosed in the above description, and in the claims and drawings can be essential for implementing the invention in its various embodiments both individually and in any combination.

1. Use of a composition consisting of or with a content of at least a dissimilated milk serum to improve the tolerance of the human or animal organism.

2. Use, in accordance with claim 1, for the treatment, prevention and prophylaxis of psoriasis, ichthyosis, atopic and orthoergic eczema, acne and etiologically similar skin disorders, and also dermatological infections, skin injuries or skin irritations.

3. Use, in accordance with claim 1, for the treatment, prevention and prophylaxis of cardiovascular diseases.

4. Use, in accordance with claim 1, for the treatment, prevention and prophylaxis of inflammatory intestinal diseases.

5. Use, in accordance with claim 4, for the treatment, prevention and prophylaxis of Crohn's disease and/or colitis.

6. Use, in accordance with claim 1, for the treatment, prevention and prophylaxis of rheumatic and rheumatoid diseases.

7. Use, in accordance with claim 1, for the treatment, prevention and prophylaxis of manic-depressive disorders.

8. Use, in accordance with claim 1, for the treatment, prevention and prophylaxis of schizophrenia.

9. Use, in accordance with any of the preceding claims, characterised in that the composition is used as an additive added to foodstuffs or animal feed.

10. Use, in accordance with any of the preceding claims, characterised in that the composition is used as to prepare a pharmaceutical composition.

11. Use, in accordance with any of the preceding claims, characterised in that the composition, is used, in the case of a medical indication, in combination, simultaneously or consecutively, with the active agents that can be employed for the indication concerned.

12. Use, in accordance with any of the preceding claims, characterised in that a milk serum which has been obtained from cow's milk, sheep's milk, goat's milk, horse milk and/or camel milk is used as the starting product for manufacturing the dissimilated milk serum.

13. Use, in accordance with claim 12, characterised in that whey is used which has been obtained as a by-product in the production of cheese, cottage cheese or yoghurt.

14. Use, in accordance with any of the preceding claims, characterised in that the dissimilated milk serum is obtained by fermentation with one or more micro-organisms selected from the group consisting of lactobacilli, leuconostoc, streptococci and other lactic acid bacteria.
